(19) United States
(12) Patent Application Publication
(10) Pub. No.: US 2023/0181851 A1
(43) Pub. Date: Jun. 15, 2023

(54) USE OF DIAPHRAGMATIC ULTRASOUND TO DETERMINE PATIENT-SPECIFIC VENTILATOR SETTINGS AND OPTIMIZE PATIENT-VENTILATOR ASYNCHRONY DETECTION ALGORITHMS

(71) Applicant: KONINKLIJKE PHILIPS N.V., EINDHOVEN (NL)

(72) Inventors: Jaap Roger Haartsen, Eindhoven (NL); Roberto Buizza, Eindhoven (NL); Joerg Sabczynski, Hamburg (DE); Rafael Wiemker, Hamburg (DE); Thomas Koehler, Hamburg (DE); Cornelis Petrus Hendriks, Eindhoven (NL); Michael Polkey, London (GB); Rita Priori, Eindhoven (NL); Nataly Wieberneit, Hamburg (DE); Stefan Winter, Aachen (DE)

(21) Appl. No.: 17/961,080

(22) Filed: Oct. 6, 2022

Related U.S. Application Data
(60) Provisional application No. 63/289,724, filed on Dec. 15, 2021.

(51) Int. Cl. A61M 16/00; G16H 20/40; G16H 40/67

(52) U.S. Cl. CPC A61M 16/024; A61M 16/0051; G16H 20/40; G16H 40/67; A61M 2230/62; A61M 2205/583

(57) ABSTRACT

A medical device for treating an associated patient includes an electronic processing device configured to receive ventilation waveform data during mechanical ventilation of the associated patient and to perform a patient-ventilator asynchrony monitoring method including detecting initial patient-ventilator asynchrony events during a training period of the mechanical ventilation by analysis of measurements of the associated patient acquired during the training period; training a machine learning (ML) component to analyze ventilation waveform data to detect patient-ventilator asynchrony events using the ventilation waveform data received during the training period with labels indicating the initial patient-ventilator asynchrony events; applying the patient-specific ML component to the ventilation waveform data received after the training period to detect patient-ventilator asynchrony events occurring after the training period; and a display device configured to display an indication of patient-ventilator asynchrony events detected by the applying of the patient-specific ML component.

### CROSS-REFERENCE TO RELATED APPLICATIONS

This patent application claims the priority benefit under 35 U.S.C. § 119(e) of U.S. Provisional Application No. 63/289,724, filed on Dec. 15, 2021, the contents of which are herein incorporated by reference.

The following relates generally to the respiratory therapy arts, mechanical ventilation arts, patient-ventilator asynchrony detection arts, machine-learning component training arts, and related arts.

### BACKGROUND

Mechanical ventilation is common in patients with conditions such as impaired lung function or respiratory failure, heart failure and in patients under general anesthesia. The purpose of mechanical ventilation is to ensure adequate oxygenation and removal of CO2 if the patient has difficulties breathing or is unable to breathe on his/her own.

Patient-ventilator asynchrony is a mismatch between the patient and the ventilator, regarding time, flow, volume, or pressure demands of the patient respiratory system (see, e.g., M. Holanda et al. “Patient-ventilator asynchrony.” J Bras Pneumol. 2018). Several types of asynchronies exist (see Table 1) from which ineffective (or wasted) effort is the most common asynchrony.

TABLE 1
List of patient-ventilator asynchronies categorized to the phase of the respiratory cycle

| Phase of | | |
|---|---|---|
| respiratory cycle | Asynchrony | Description |
| Beginning of | Delayed | Delay between inspiratory effort and delivered breath |
| | triggering | |
| inspiration | Ineffective | Patient’s inspiratory effort fails to trigger mechanical |
| | effort | breath |
| | Auto triggering | Delivery of mechanical breath without inspiratory |
| | | effort |
| During gas delivery | Flow | Delivered flow doesn’t meet demand |
| | asynchrony | |
| End of inspiration | Double | Multiple delivered breaths during one inspiratory |
| | triggering | effort |
| | Early cycling | Duration of delivered breath is shorter than |
| | | inspiratory effort |
| | Delayed cycling | Duration of delivered breath is longer than |
| | | inspiratory effort |

Patient-ventilator asynchronies may cause discomfort, anxiety, increased work of breathing, and worsening of gas exchange. Approximately 25% of ICU patients receiving pressure support ventilation (PSV) suffer from a high incidence of asynchronies (see, e.g., E. Garofalo et al. “Recognizing, quantifying and managing patient-ventilator asynchrony in invasive and noninvasive ventilation.” Expert Rev Respir Med. 2018). This number can be as high as 80% for patients diagnosed with, for example, neuromuscular disease, chronic respiratory failure, or chronic obstructive pulmonary disease (COPD) receiving long-term non-invasive ventilation at home (see, e.g., M. Ramsay et al. “Parasternal electromyography to determine the relationship between patient-ventilator asynchrony and nocturnal gas exchange during home mechanical ventilation set-up.” Thorax 2015). The rate of asynchronies is quantified by the asynchrony index (AI %) as the ratio of the number of asynchronous breaths and the total breath count, expressed as a percentage. Values of AI %>10% are associated with a negative impact on patient outcomes such as increased weaning failures, longer duration of mechanical ventilation, and higher ICU and hospital mortality. Hence, it is important to select appropriate ventilator settings to keep the rate of asynchronies to a minimum. Furthermore, it has been shown that the incidence of asynchronies varies over time; in particular, ineffective efforts tend to occur in clusters between uneventful periods. It is therefore important to continuously detect and quantify patient-ventilator asynchronies to continuously assess appropriateness of ventilator settings.

Various asynchrony detection methods are used during invasive ventilation. The most important ones are based on visual analysis of the ventilator waveforms, as well as on the use of specific software, assessment of the electrical activity of the diaphragm (i.e., EAdi), and measurement of esophageal or transdiaphragmatic pressure.

Assessment of the electrical activity of the diaphragm EAdi or esophageal (or transdiaphragmatic) pressure is considered the gold standard for detection of patient-ventilator asynchronies as it allows for a good estimation of inspiratory effort. However, both methods are semi-invasive as it requires placement of a dedicated nasogastric feeding tube with a distal array of multiple electrodes (EAdi) or a balloon-tipped catheter (esophageal and transdiaphragmatic pressure). Furthermore, EAdi monitoring requires a specific ventilator equipped with technology and software. Additional pressure measurements require the placement and calibration of balloon-tipped catheters, which is quite complex to accomplish. For these reasons, widespread use of EAdi and esophageal pressure for patient-ventilator detection is limited. In addition, because of its semi-invasive character it is not used in long-term non-invasive ventilation at home.

Visual monitoring of ventilator pressure and flow waveforms at the bedside is better accessible than EAdi or esophageal pressure monitoring and is therefore the most frequently used method to detect patient-ventilator asynchronies. However, this method is not very accurate, objectively difficult, and requires training of clinicians. According to one study (see, e.g., D. Colombo et al. “Efficacy of ventilator waveforms observation in detecting patient-ventilator asynchrony.” Critical care medicine 2011), sensitivity for detecting patient-ventilator asynchronies in invasively ventilated patients by trained clinicians is only 28%. For non-invasive ventilation sensitivities as low as 20% are reported (see, e.g., F. Longhini et al. “Efficacy of ventilator waveform observation for detection of patient-ventilator asynchrony during NIV: A multicentre study.” ERJ Open Research 2017).

Algorithms for automatic detection of patient-ventilator asynchronies exist (see, e.g., L. Blanch et al. “Validation of the Better Care® system to detect ineffective efforts during expiration in mechanically ventilated patients: a pilot study.” Intensive Care Med. 2012; Q. Mulqueeny et al. “Automatic detection of ineffective triggering and double triggering during mechanical ventilation.” Intensive Care Med. 2007; G. Gutierrez et al. “Automatic detection of patient-ventilator asynchrony by spectral analysis of airway flow.” Crit Care. 2011; and M. Younes et al. “A method for monitoring and improving patient: ventilator interaction.” Intensive Care Med. 2007), and allow for non-invasive automated quantification of patient-ventilator asynchronies. In general, these algorithms are more accurate than bedside visual analysis of the ventilator pressure and flow waveforms but less accurate than EAdi and esophageal pressure monitoring. Such algorithms also typically do not consider inter-patient variation that can be expected to be present amongst the diverse population of patients receiving mechanical ventilation for a wide range of medical issues.

Diagnostic ultrasound is a noninvasive imaging modality that is readily available in the ICU, and can also be used to detect patient-ventilator asynchronies. Diaphragmatic ultrasonography, a subclass of diagnostic ultrasound, allows for the visualization of the diaphragm (see, e.g., P. Tuinman et al. “Respiratory muscle ultrasonography: methodology, basic and advanced principles and clinical applications in ICU and ED patients-a narrative review.” Intensive Care Med. 2020). Two approaches exist: the mid-axillary intercostal approach at the zone of apposition and the subcostal approach using the liver or spleen as an acoustic window.

The intercostal approach is performed with a 10-15 MHz linear array transducer and allows for real-time measurement of the diaphragm thickness. The diaphragm thickens with active shortening and, therefore, thickening fraction (TF) reflects contractile activity. The thickening fraction of the diaphragm (TFdi) is calculated in B-mode or M-mode as the percentage inspiratory increase in the diaphragm thickness relative to end-expiratory thickness (Tee) during tidal breathing, according to Equation 1:

$$TFdi = \frac{{T}_{ei} - {T}_{ee}}{{T}_{ee}} * 100 \% \qquad ( 1 )$$

where Tₑᵢ the end-inspiratory thickness.

Some studies (see, e.g., E. Vivier et al. “Diaphragm ultrasonography to estimate the work of breathing during non-invasive ventilation.” Intensive Care Med. 2012; M. Umbrello et al. “Diaphragm ultrasound as indicator of respiratory effort in critically ill patients undergoing assisted mechanical ventilation: a pilot clinical study.” Crit Care. 2015) have evaluated the correlation between TFdi and respiratory effort. In another study (see, e.g., E. Oppersma et al. “Functional assessment of the diaphragm by speckle tracking ultrasound during inspiratory loading.” J Appl Phys. 2017), the functional assessment of the diaphragm was studied by speckle tracking ultrasound during inspiratory loading. The technique of speckle tracking ultrasound allows for the detection and tracking of anatomic structure deformation such as the diaphragm over time by analyzing acoustic markers called speckles. Both diaphragm strain and diaphragm strain rate were highly correlated to transdiaphragmatic pressure Pdi (strain r²=0.72; strain rate r²=0.80) and EAdi (strain r²=0.60; strain rate r²=0.66) and concluded that speckle tracking ultrasound is superior to ultrasound TFdi measurements.

Diaphragmatic excursion is measured with a 2-5 MHz phased-array or curved-array (“abdominal”) probe positioned just below the costal arch at the midclavicular line. Excursion is quantified in M-mode, with the M-line placed perpendicular to the direction of motion. Although active contraction of the diaphragm cannot be distinguished from passive displacement, due to ventilator inspiratory pressures it still can be used to identify e.g. ineffective efforts.

Lung sliding is the sliding back and forth of the visceral and parietal pleura on one another as the patient breathes. Lung sliding can be assessed qualitatively by ultrasound at the anterior, lateral, and posterior chest and is mainly used for the diagnosis of pneumothorax. Recent advances in ultrasound image processing such as speckle tracking allow for quantification of lung sliding (see, e.g., G. Duclos et al. “Speckle tracking quantification of lung sliding for the diagnosis of pneumothorax: A multicentric observational study.” Intensive Care Med. 2019; G. Duclos et al. “A picture's worth a thousand words: Speckle tracking for quantification and assessment of lung sliding.” Intensive care med. 2019; E. Fissore et al. “Pneumothorax diagnosis with lung sliding quantification by speckle tracking: A prospective multicentric observational study.” The American journal of emergency medicine 2021; and L. Crognier et al. “Diaphragmatic speckle tracking imaging for 2D-strain assessment in mechanical ventilation weaning test.” Medical hypotheses 2021).

As seen in the foregoing review, several patient-ventilator asynchrony detection methods exist. However, these methods have several drawbacks. For example, EAdi and esophageal (or transdiaphragmatic) pressure assessment are considered the gold standard for accurate continuous detection of patient-ventilator asynchronies. However, these methods are semi-invasive and require a specific ventilator equipped with patented technology and software or are complex to accomplish. Therefore, widespread use in the ICU is limited and these methods are not used in long-term non-invasive ventilation at home.

Visual monitoring of ventilator pressure and flow waveforms at the bedside is better accessible than EAdi or esophageal pressure monitoring. However, this method is not very accurate, objectively difficult, requires training of clinicians and is not continuous such that occurrence of asynchronies may be over- or underestimated.

Automated algorithms that analyze ventilator pressure and flow waveforms for automatic continuous detection of patient-ventilator asynchronies are noninvasive and better accessible than EAdi and esophageal pressure monitoring. These algorithms are more accurate than bedside visual analysis of the ventilator waveforms but less accurate than EAdi and esophageal pressure monitoring. However, such algorithms do not consider inter-patient variation amongst the diverse population of patients receiving mechanical ventilation for a wide range of medical issues.

Diaphragmatic and lung sliding ultrasound are better accessible techniques than EAdi and esophageal pressure measurement and can be used for non-invasive assessment of patient-ventilator asynchronies. However, these techniques generally cannot be used continuously throughout the ICU stay.

The following discloses certain improvements to overcome these problems and others.

### SUMMARY

In one aspect, a medical device for treating an associated patient includes an electronic processing device configured to receive ventilation waveform data during mechanical ventilation of the associated patient and to perform a patient-ventilator asynchrony monitoring method including detecting initial patient-ventilator asynchrony events during a training period of the mechanical ventilation by analysis of measurements of the associated patient acquired during the training period; training a machine learning (ML) component to analyze ventilation waveform data to detect patient-ventilator asynchrony events using the ventilation waveform data received during the training period with labels indicating the initial patient-ventilator asynchrony events, the trained ML component forming a patient-specific ML component that is specific to the associated patient; applying the patient-specific ML component to the ventilation waveform data received after the training period to detect patient-ventilator asynchrony events occurring after the training period; and a display device (14) configured to display an indication of patient-ventilator asynchrony events detected by the applying of the patient-specific ML component.

In another aspect, a mechanical ventilation method includes receiving ventilation waveform data during mechanical ventilation of an associated patient; detecting initial patient-ventilator asynchrony events during a training period of the mechanical ventilation by analysis of measurements of the associated patient acquired during the training period; training a ML component to analyze ventilation waveform data to detect patient-ventilator asynchrony events using the ventilation waveform data received during the training period with labels indicating the initial patient-ventilator asynchrony events, the trained ML component forming a patient-specific ML component that is specific to the associated patient; applying the patient-specific ML component to the ventilation waveform data received after the training period to detect patient-ventilator asynchrony events occurring after the training period; and displaying an indication of patient-ventilator asynchrony events detected by the applying of the patient-specific ML component.

One advantage resides in providing continuous monitoring for patient-ventilator asynchrony in a non-invasive manner.

Another advantage resides in providing such continuous monitoring by performing ultrasound for diaphragmatic or lung sliding for a predetermined at a start of ventilation therapy for a patient to train a machine-learning patient-ventilator asynchrony detection algorithm which can subsequently be used for continuous non-invasive patient-ventilator asynchrony monitoring.

Another advantage resides in providing continuous patient-ventilator asynchrony monitoring that can be optimized for different postures of the patient.

Another advantage resides in using a trained machine-learning patient-ventilator asynchrony detection algorithm to detect asynchronies only using ventilator input signals from a mechanical ventilator delivering ventilation therapy to a patient.

Another advantage resides in non-invasive, easily accessible and accurate method for continuous detection of patient-ventilator asynchronies.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative mechanical ventilation system in accordance with the present disclosure.

FIG. 2 shows an example flow chart of operations suitably performed by the system of FIG. 1.

### DETAILED DESCRIPTION

As used herein, the singular form of “a”, “an”, and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

In various embodiments disclosed herein, improved patient-ventilator asynchrony monitoring is disclosed, which is noninvasive and provides continuous monitoring. In some illustrative embodiments, initial patient-ventilator asynchrony events are detected during a training period of the mechanical ventilation. This is done by analysis of second modality measurements of the patient acquired during the training period using a second modality, such as ultrasound. A machine learning (ML) component is then trained to analyze ventilation waveform data to detect patient-ventilator asynchrony events. The training uses ventilation waveform data received during the training period and labeled with the initial patient-ventilator asynchrony events from the second modality measurement analysis. The trained ML component thus forms a patient-specific NIL component that is specific to the specific patient undergoing mechanical ventilation. The patient-specific ML component is applied to the ventilation waveform data received after the training period to detect patient-ventilator asynchrony events occurring after the training period.

The training period typically corresponds to an initial period of the mechanical ventilation, for example with the ultrasound or other second modality monitoring being applied immediately after the patient is placed onto or otherwise connected to the mechanical ventilator. This is convenient as the patient is already likely to be being closely monitored during this initial period. The training period can be relatively brief, for example in a range of 1 minute to 20 minutes inclusive in some non-limiting embodiments. Additionally or alternatively, the training period can be continued until some minimum number of initial patient-ventilator asynchrony events are detected, so as to provide a sufficient number of events for training the ML component.

Since the ultrasound or other second modality patient measurements are noninvasive, the training period can advantageously be repeated if conditions of the mechanical ventilation change, in order to provide further data for update-training the ML component. For example, if the patient's physician changes the ventilation mode, or the patient's condition improves or deteriorates, then the training period can be repeated to adjust the ML component to account for those changed conditions. In another contemplated variant, a training period can be used for each of two or more different patient postures (e.g., lying down or sitting in a wheelchair) and different versions of the ML component can be trained for the different patient postures. This can be beneficial since patient-ventilator asynchrony can be patient posture-dependent.

In further variant embodiments, the system can propose ventilator setting adjustment(s) based on detected patient-ventilator asynchrony events. In further variant embodiments, the system can be operatively connected to automatically execute such ventilator setting adjustment(s).

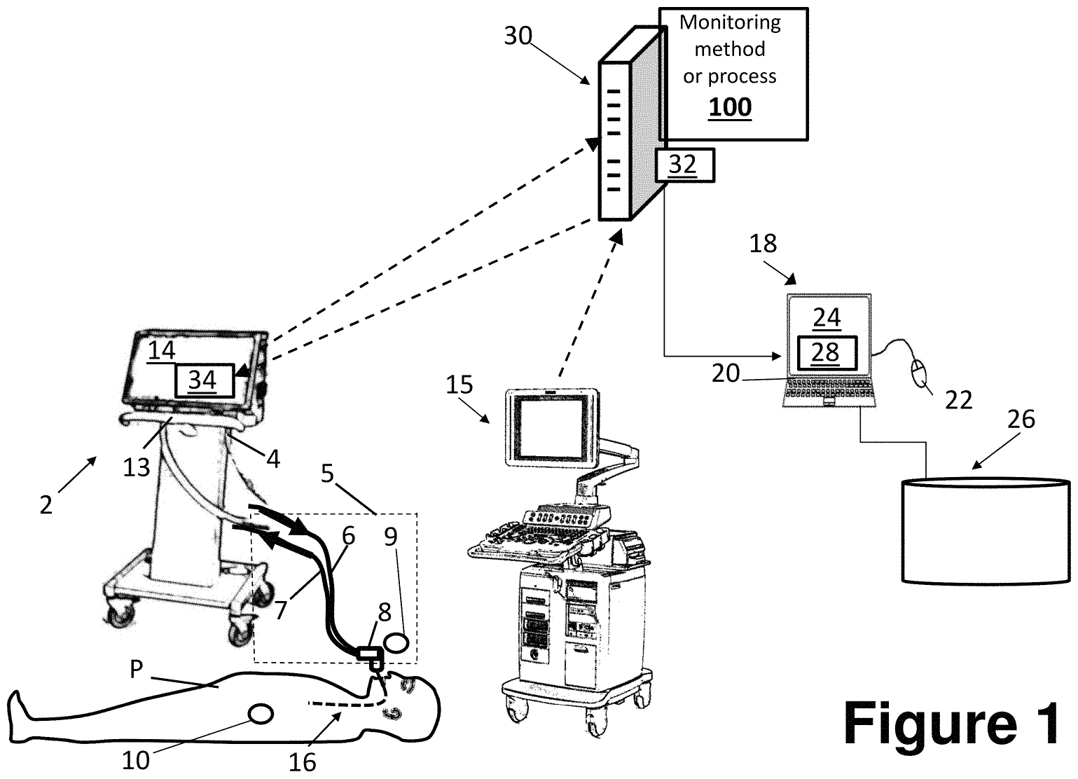

With reference to FIG. 1, a medical device including a mechanical ventilator 2 for providing ventilation therapy to an associated patient P is shown. As shown in FIG. 1, the mechanical ventilator 2 includes an outlet 4 connectable with a patient breathing circuit 5 to deliver mechanical ventilation to the patient P. The patient breathing circuit 5 includes typical components for a mechanical ventilator, such as an inlet line 6, an optional outlet line 7 (this may be omitted if the ventilator employs a single-limb patient circuit), a connector or port 8 for connecting with an endotracheal tube (ETT), and one or more breathing sensors 9, such as a gas flow meter, a pressure sensor (such as a central venous pressure (CVP) sensor), an air flow sensor end-tidal carbon dioxide (etCO₂) sensor, and/or so forth. A posture sensor 10 (e.g., an accelerometer, a camera, and so forth) can also be included to detect a posture of the patient P as a function of time during the mechanical ventilation. The mechanical ventilator 2 is designed to deliver air, an air-oxygen mixture, or other breathable gas (supply not shown) to the outlet 4 at a programmed pressure and/or flow rate to ventilate the patient via an ETT. The mechanical ventilator 2 also includes an electronic controller (e.g., a microprocessor) 13 for controlling operation of the mechanical ventilator 2, and a display device 14 for displaying information about the patient P and/or settings of the mechanical ventilator 2 during mechanical ventilation of the patient P.

FIG. 1 diagrammatically illustrates the patient P intubated with an ETT 16 (the lower portion of which is inside the patient P and hence is shown in phantom). Other patient couplings are contemplated, such a noninvasive full-face mask or coupling via a tracheotomy. The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 1 also shows a medical imaging device 15 (also referred to as an image acquisition device, imaging device, and so forth). The image acquisition device 15 can be a Computed Tomography (CT) image acquisition device, a C-arm imager, or other X-ray imaging device; Magnetic Resonance (MR) image acquisition device; an ultrasound (US) image acquisition device; or a medical imaging device of another modality. As primarily described herein, the medical imaging device 15 comprises a US medical imaging device 15. For monitoring patient-ventilator asynchrony, the ultrasound transducer (not shown) of the US imaging device 15 may for example be placed in a mid-axillary intercostal position at the zone of apposition, or in a subcostal position using the liver or spleen as an acoustic window. It should be noted that the imaging device 15 may not be located in the same room, or even the same department, as the mechanical ventilator 2. For example, the medical imaging device 15 may be located in a radiology laboratory while the mechanical ventilator 2 may be located in an intensive care unit (ICU), cardiac care unit (CCU), in a hospital room assigned to the patient P, or so forth. This is diagrammatically indicated in FIG. 1 by separator line L. Additionally or alternatively, a bedside imaging device 15, such as the illustrative ultrasound imaging device, may be used. For various reasons, the medical imaging device 15 may not be available or usable for continuous monitoring of patient-ventilator asynchrony in the patient P. For example, the ICU may have only a single ultrasound imaging device which cannot be dedicated to a single patient for an extended period. CT, MR, C-arm imagers and the like are expensive and hence also cannot be dedicated to monitoring a single patient for an extended time period. As disclosed herein, however, this is not an issue as the medical imaging device 15 is only used for a relatively brief training period (e.g., around 120 minutes in some nonlimiting embodiments) to collect training data for training a ML component to thereafter monitor patient-ventilator asynchrony via waveforms of the mechanical ventilator 2.

Alternatively, instead of imaging data acquired by the imaging device 15, the sensor 9 can measure measurements sensitive to patient-ventilator asynchrony (e.g., via a catheter (not shown) to measure central venous pressure (CVP), or the sensor 9 can comprise an electromyography (EMG) sensor to measure parasternal activity of abdominal muscles, an esophageal sensor, a sensor to measure EAdi, and so forth) which may be used to provide a signal indicative of the respiratory muscle activities.

With continuing reference to FIG. 1, an electronic processing device 18 configured to generate data related to the patient P and/or settings of the mechanical ventilator 2 during mechanical ventilation of the patient P is shown. The electronic processing device 18 can comprise an electronic processing device, such as a workstation computer (more generally, a computer), a smart device (e.g., a smartphone, a tablet, and so forth), or server computer or a plurality of server computers, (e.g., interconnected to form a server cluster, cloud computing resource, or so forth). The electronic processing device 18 includes typical components, such as an electronic controller 20 (e.g., an electronic processor or a microprocessor), at least one user input device (e.g., a mouse, a keyboard, a trackball, a finger swipe on a touchscreen of a smart device, and/or the like) 22, and at least one display device 24 (shown only in FIG. 1, e.g., an LCD display, plasma display, cathode ray tube display, and/or so forth). In some embodiments, the display device 24 can be a separate component from the electronic processing device 18. The display device 24 may also comprise two or more display devices. While the electronic processing device 18 is shown as a separate device from the mechanical ventilator 2, in other embodiments it is contemplated for the electronic processing device 18 to comprise the electronic processor of the mechanical ventilator 2. Said another way, it is contemplated for the electronic processing device 18 to be integrated with the mechanical ventilator 2.

The electronic controller 20 is operatively connected with one or more non-transitory storage media 26. The non-transitory storage media 26 may, by way of nonlimiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof, or so forth; and may be for example a network storage, an internal hard drive of the ventilation assistance device 18, various combinations thereof, or so forth. It is to be understood that any reference to a non-transitory medium or media 26 herein is to be broadly construed as encompassing a single medium or multiple media of the same or different types. Likewise, the electronic controller 20 may be embodied as a single electronic processor or as two or more electronic processors. The non-transitory storage media 26 stores instructions executable by the at least one electronic controller 20. The instructions include instructions to generate a graphical user interface (GUI) 28 for display on the display device 24.

In some embodiments, a cloud server computer 30 (shown in FIG. 1 as a server computer) is in communication with the mechanical ventilator 2 and the imaging device 15, and stores executable instructions to perform a patient-ventilator asynchrony monitoring method or process 100 to provide ventilation therapy to the patient P. In another example, an electronic processing device (such as an edge device or a hub, which is not shown in FIG. 1) is configured to collect and synchronize ventilation waveforms and data from the sensor 9 and/or the imaging device 15. The cloud server computer 30 can serve as the electronic processing device 18 alone or in combination with a local implementation of the electronic processing device 18 (e.g., as the illustrative separate device or as integrated with the mechanical ventilator 2). For example, the cloud server computer 30 can perform computationally complex tasks such as training the machine learning component.

As described herein, the patient-ventilator asynchrony monitoring method 100 can be performed by the electronic processing device 18, or can be performed by the electronic controller 13 of the mechanical ventilator 2.

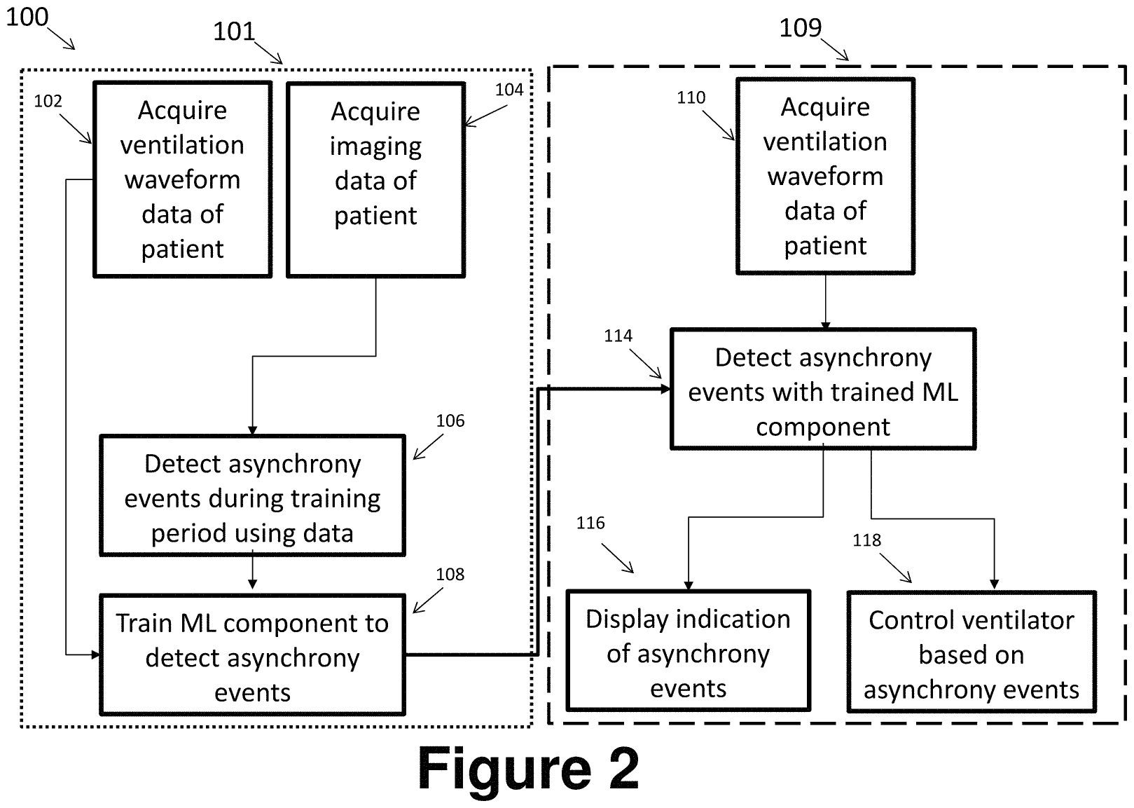

With reference to FIG. 2, and with continuing reference to FIG. 1, an illustrative embodiment of the patient-ventilator asynchrony monitoring method 100 is diagrammatically shown as a flowchart. Initially, the patient-ventilator asynchrony monitoring method 100 includes a training phase 101. At an operation 102, ventilation waveform data comprising at least one of ventilation pressure data and/or ventilation air flow data is measured by the sensor 9, during the mechanical ventilation is acquired by the mechanical ventilator 2. The ventilation pressure data and/or ventilation air flow data can be referred to as a first modality measurement. The ventilation pressure data and/or ventilation air flow data can be transmitted to the cloud server 30 and/or the electronic processing device 18.

At an operation 104, second modality measurements of the associated patient are acquired during a training period using a second modality. In the illustrative example, the second modality is ultrasound and the operation 104 acquires ultrasound imaging data (e.g., B-mode, or M-mode, and so forth) of the patient is acquired using the US image acquisition device 15. The ultrasound imaging data can comprise imaging of the diaphragm of the patient P, accessory inspiratory muscles (i.e. abdominal, intercostal, etc.) of the patient P, or lung sliding of the patient P undergoing mechanical ventilation. In other embodiments, another non-invasive second modality that is sensitive to patient-ventilator asynchrony may be used, such as measurements of parasternal electromyography (EMG) activity of abdominal muscles. The imaging data can be transmitted to the cloud server 30.

At an operation 106, initial patient-ventilator asynchrony events are detected during a training period of the mechanical ventilation by analysis of the second modality measurements of the patient P, for example by analysis of diaphragm thickening or lung sliding in the case of US measurements, or analysis of EMG activity of abdominal muscles. The second modality measurements are acquired during the training period in the second modality measurement operation 104. The training period is in a range of 1 minute to 20 minutes. The patient-ventilator asynchrony event detection operation 106 is performed by the cloud server 30 or by the electronic processing device 18.

At an operation 108, a machine learning (ML) component 32 (implemented in the cloud server 30 or the mechanical ventilator 2) is trained (i.e., using a large data set of asynchrony events) to detect patient-ventilator asynchrony events using the ventilation waveform data from the ventilation waveform data acquisition operation 102 labeled with patient-ventilator asynchrony events identified in the operation 106 from the imaging data acquired during the image acquisition operation 104. For example, in some embodiments the ML component 32 is an existing algorithm for automatic detection of patient-ventilator asynchrony events with one or more thresholds or other parameters of the algorithm optimized for the patient P by the training. The ML component 32 forms a patient-specific ML component that is specific to the patient P. The training operation 108 trains the ML component 32 to analyze the ventilation waveform data that are labeled with the initial patient-ventilator asynchrony events from the initial patient-ventilator asynchrony events detection operation 106.

In some embodiments, multiple versions of the ML component 32 may be trained for different postures of the patient P. To do so, the training phase 101 is repeated with the patient in different postures (e.g., sitting, laying down) to generate labeled training data for each posture, which is used to train patient-specific and posture-specific ML components 32 for the different postures. Similarly, the training phase 101 can be repeated any time there is a significant change in the patient's situation, such as improvement or deterioration of the patient's condition, a change in the mode of mechanical ventilation therapy, a material change in some other therapy also being administered to the patient (e.g., if the patient is placed on a new drug or taken off a previous drug that may be reasonably expected to affect the patient's respiratory system).

To train the ML component 32, a machine learning asynchrony detection algorithm implemented in the ML component 32 is trained using the labelled ventilator waveforms. Alternative optimization methods may be used. For example, in the case of an algorithm that detects ineffective efforts through the analysis of flow and airway pressure deflections the optimal trigger threshold level is determined such that the amount of detected ineffective efforts is maximal (which is achieved by lowering the threshold level for higher sensitivity) while the amount of auto-triggers is minimal (avoiding the threshold level being too low). In case no asynchronies occurred in the training period (i.e., 1-10 minutes) predetermined threshold levels could be used that are optimal values for a group of patients. The design of the algorithm may be such that it allows for quantification of patient-ventilator interaction (e.g., to quantify delays between onset of diaphragm strain, thickening or excursion and pressure support). The detection algorithm is not limited to ineffective efforts only but can be broader such as to detect other types of asynchronies as well. The ventilation waveform data and the imaging data may be split into a training, validation, and test set to allow for (intermediate) validation and (final) testing of the optimized model.

In some embodiments of the training phase 101, the mechanical ventilator 2 could automatically change its settings, or suggest that a medical professional apply new settings to facilitate the appearance of asynchrony events to enrich the data used in the training phase 101. The mechanical ventilator 2 and the US imaging device 15 can share a common clock to ensure accurate synchronization of the acquired data. Alternatively, the cloud server 30 (or edge device or hub) can be programmed to synchronize the signal between the ventilator data and the ultrasound imaging data.

Furthermore, while a single ML component 32 is described here, more generally multiple classification algorithms (i.e., multiple ML components) may be trained to detect various types of patient-ventilator asynchronies. For example, one ML component may be trained to detect delayed triggering, another to detect flow asynchrony, another to detect early cycling, and/or so forth. This enables each ML component to be optimized for a specific type of patient-ventilator asynchrony. For any ML component where the training data includes no labeled events of that particular type of patient-ventilator asynchrony, a default value or values for the optimizable parameters of that ML component can be used, or alternatively that ML component can be left unused under the assumption the patient is not suffering from that type of patient-ventilator asynchrony. Still further, multiple parameters may be used as input to the classification algorithms (e.g., diaphragm excursion, thickness, auxiliary muscle activities, EMG, ventilation settings of the mechanical ventilator 2, and so forth). Next to classification, the ultrasound time-series data and ventilator waveforms may be used for quantification of patient-ventilator interaction, e.g., to quantify delays between onset of diaphragm strain, thickening or excursion and pressure support. The ventilator waveform data and the imaging data can then be labelled.

The ultrasound imaging data is processed by the cloud server 30 and/or the ultrasound imaging device 15 to extract time-series data such as diaphragm excursion, diaphragm thickness, diaphragm strain or strain rate, accessory inspiratory muscle activity or lung sliding. The ultrasound imaging data may be pre-processed for noise reduction using known techniques such as autoencoders. Techniques for feature detection such as the diaphragm or pleural line can also be used, such as computer vision (CV) techniques like edge detection or convolutional neural networks (CNNs) trained for detection of the diaphragm or pleural line. Also, techniques for quantification of displacement of regions of interest on the detected features, such as determining the cross-correlation of regions of interest between various frames, can be used. Finally, time-series data may be filtered. The processed ultrasound time-series data and simultaneously acquired ventilator pressure and flow waveforms are uploaded to the cloud server 30 (when processed by the imaging device 15) or locally stored on imaging device 15 and/or the mechanical ventilator 2.

The ventilator waveforms may be uploaded to the cloud server 30 directly from the mechanical ventilator 2 (or to the edge device or hub), which can advantageously eliminate the need for a connection between the US imaging device 15 and the mechanical ventilator 2. To this end, it is essential that the clocks of the US imaging device 15 and the mechanical ventilator 2 are synchronized and that the processed ultrasound time-series data and ventilator data contain a time stamp to facilitate data synchronization in the cloud server 30. Alternatively, a landmark is created, e.g. by shortly in- or decreasing the applied pressure which is detectable in the ventilator waveforms and processed ultrasound time-series data to facilitate data synchronization.

Once the ML component 32 is trained, the training phase 101 is complete, the ultrasound, EMG, or other second modality measurement device can be disconnected from the patient P, and the patient-ventilator asynchrony monitoring method 100 proceeds to an application phase 109 in which patient-ventilator asynchrony events are detected using only the ventilation waveform data and/or ventilation settings of the mechanical ventilator 2. At an operation 110, ventilation waveform data of the patient P is acquired by the mechanical ventilator 2 and transferred to the cloud server 30. At an operation 114, the trained patient-specific ML component 32 is applied to the ventilation waveform data acquired during the ventilation waveform data acquisition operation 110 to detect patient-ventilator asynchrony events occurring during mechanical ventilation therapy of the patient P.

To do so, the ventilation waveform data acquired at the operation 110 are processed by the ML component 32 to detect and classify patient-ventilator asynchronies (e.g., by feeding it into the classification algorithm implemented in the ML component 32 and trained during the training phase 101). If multiple classification algorithms for different types of patient-ventilator asynchronies were trained in the training phase 101, then the operation 114 may apply the various trained classification algorithms to detect the corresponding various types of patient-ventilator asynchronies.

At an operation 116, an indication 34 of patient-ventilator asynchrony events detected by the patient-specific ML component 32 can be displayed on the display device 14 of the mechanical ventilator 2. In some embodiments, the indication 34 of patient-ventilator asynchrony events comprises an asynchrony index, which is displayed on the display device 14. The asynchrony index can be calculated by the cloud server 30 as a ratio of a number of asynchronous breaths of the patient P in the labelled waveforms and a total breath count of the patient P. The asynchrony index (AI %) is displayed as a number on the display device 14, displaying the ratio of the number of asynchronous breaths of all types of asynchronies and the total breath count, expressed as a percentage. In some examples, the AI % can be shown as an index over a predetermined time period (i.e., an hour or so). In addition, asynchrony specific indices such as ineffective triggering index (ITI %) may be displayed. Also, the asynchrony and asynchrony specific indices may be calculated over a predetermined time interval (e.g., the previous hour or two) and displayed as function of time during the predetermined time interval. Finally, a clustering index or volatility may be calculated and displayed on the display device 14. For example, this could be determined by calculating the standard deviation of the asynchrony index time-series data.

In other embodiments, the indication 34 can include a message comprising a proposed adjustment to the mechanical ventilation device 2 to reduce or eliminate the patient-ventilator asynchrony events, and the proposed adjustment can be displayed on the display device 14. In other embodiments, the indication 34 can include one or more optimized settings of the mechanical ventilator 2, which can be determined based on the detected patient-ventilator asynchrony events (and optionally also on the received imaging data), and the one or more optimized settings can be displayed on the display device 14. These are merely examples and should not be construed as limiting.

Optimal ventilation settings can be chosen based on the information provided in Table 2 (below) or other similar compilation of remedial actions. Two main types of asynchronies can include auto-triggering and ineffective triggering, but the optimized algorithm can be able to detect all the asynchronies because the ML component 32 was trained using ground truth data. The ventilator settings optimization may be based on trial and error, (i.e., evaluate the effect of a proposed solution and in case the targeted reduction in AI % is not met, then proceed to the next solution). The ventilation settings optimization algorithm may be self-learning to improve over time. The algorithm to determine the optimal settings may run in the cloud server 30, on the US imaging device 15, the mechanical ventilator 2, or on a separate processing unit (i.e., the electronic processing device 18).

TABLE 2
Solutions to reduce auto-triggering and ineffective effort type of asynchronies

| | | | Lab |
|---|---|---|---|
| | | | solution |
| Asynchrony | Cause | Solutions | testing |
| Auto | 1. High trigger sensitivity | 1. Adjust trigger sensitivity | 1. Adjust |
| triggering | 2. Leaks | 2. Reduce noise | trigger |
| | 3. Random noise in the circuit (e.g., | 3. Remove leaks | sensitivity |
| | cardiac oscillations, | 4. Use appropriate non-invasive | (priority) |
| | condensed water in the ventilator | ventilation (NIV) software | 2. Cases |
| | circuit, copious | 5. Remove circuit condensate | with and |
| | tracheobronchial secretions) | | without |
| | | | water in |
| | | | tube (not a |
| | | | priority) |
| Ineffective | 1. Low trigger sensitivity | 1. Adjust trigger sensitivity | 1. Adjust |
| efforts | 2. Weak respiratory drive or weak | 2. Reduce sedation or use drugs | trigger |
| | effort secondary to | with no effect on the respiratory | sensitivity |
| | heavy sedation, excessive respiratory | drive | 2. Increase |
| | support, or | 3. Reduce support | PEEP (in |
| | diaphragm dysfunction | 4. Correct metabolic alkalosis | case of |
| | 3. Presence of high threshold load | 5. Increase PEEP to counter | iPEEP) |
| | such as intrinsic PEEP (air trapping) | intrinsic PEEP | 3. Increase |
| | 4. Delayed cycling, especially in PSV | 6. Shorten inspiratory time | Cycling |
| | mode or | 7. Adjust end-expiratory trigger | 4. Decrease |
| | obstructive condition (FIG. 2), or | criteria in an obstructive | PSV |
| | during NIV in | condition (e.g., COPD) | |
| | presence of intentional leak in a | 8. Use appropriate NIV | |
| | ventilator unable to | software, | |
| | compensate for them | 9. Consider a neural trigger if | |
| | 5. Inspiratory time too long in a time- | the problem persists. | |
| | cycled breath (related to 4) | 10. Check for dyspnea | |
| | 6. Low level of pCO2 (related to 2) | 11. Decrease PSV level (PSV | |
| | | model) | |

Sources:
Lucia Mirabella, Gilda Cinnella, Roberta Costa, Andrea Cortegiani, Livio Tullo, Michela Rauseo, Giorgio Conti, Cesare Gregoretti Patient-Ventilator Asynchronies: Clinical Implications and Practical Solutions Respiratory Care Jul 2020, respcare.07284; DOI: 10.4187/respcare.07284;
de Haro, C., Ochagavia, A., Lopez-Aguilar, J. et al. Patient-ventilator asynchronies during mechanical ventilation: current knowledge and research priorities. ICMx 7, 43 (2019).
https://doi.org/10.1186/s40635-019-0234-5;
Subira C, de Haro C, Magrans R, Fernandez R, Blanch L. Minimizing Asynchronies in Mechanical Ventilation: Current and Future Trends. Respir Care. 2018 Apr;63(4):464-478. doi: 10.4187/respcare.05949. Epub 2018 Feb 27. Erratum in: Respir Care. 2019Mar;64(3):e1. PMID: 29487094.;
Holanda MA, Vasconcelos RDS, Ferreira JC, Pinheiro BV. Patient-ventilator asynchrony [published correction appears in J Bras Pneumol. 2018 Sep 03;]. J Bras Pneumol. 2018;44(4):321-333. doi:10.1590/S1806-37562017000000185

At an operation 118, the mechanical ventilator 2 can be controlled by adjusting settings of the mechanical ventilator 2 with the determined one or more optimized settings. In some embodiments, the optimized patient-ventilator detection algorithm (implemented in the ML component 32) can track the progression of the patient-ventilator interaction to predict the possibility of future asynchronies. The output of such prediction can be used directly to optimize further the ventilation settings, or it can be used to alert the medical professionals. The ML component 32 has features that are representative of the patient's physiological state and capture the reasons of the asynchrony. Once the ML component 32 has these interpretable features it can track the progression and predict the appearance of asynchronies. Once the ML component 32 is able to predict asynchronies it can also alarm the caregivers to take preventive actions to avoid the asynchronies or to plan their activities accordingly. The ML component 32 can be optimized using the same patient data, or/and population data.

In some embodiments, the sensor 9 can include esophageal pressure, Central Venous Pressure (CVP), EAdi or other respiratory effort indicators may be used in addition to, or instead of ultrasound imaging data as ground truth to train the ML component 32.

In some embodiments, hybrid modelling for faster training of the ML component 32 can be performed with less data (e.g. combination of a biophysical model with machine learning).

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A medical device for treating an associated patient, comprising:
an electronic processing device configured to receive ventilation waveform data during mechanical ventilation of the associated patient and to perform a patient-ventilator asynchrony monitoring method including:
detecting initial patient-ventilator asynchrony events during a training period of the mechanical ventilation by analysis of measurements of the associated patient acquired during the training period;
training a machine learning (ML) component to analyze ventilation waveform data to detect patient-ventilator asynchrony events using the ventilation waveform data received during the training period with labels indicating the initial patient-ventilator asynchrony events, the trained ML component forming a patient-specific ML component that is specific to the associated patient;
applying the patient-specific ML component to the ventilation waveform data received after the training period to detect patient-ventilator asynchrony events occurring after the training period; and
a display device configured to display an indication of patient-ventilator asynchrony events detected by the applying of the patient-specific ML component.

2. The device of claim 1, wherein the measurements comprise diaphragmatic or lung sliding ultrasound measurements or parasternal electromyography (EMG) measurements or central venous pressure (CVP) measurements.

3. The device of claim 1, wherein the measurements comprise noninvasive measurements.

4. The device of claim 1, wherein the training period is in a range of 1 minute to 20 minutes inclusive.

5. The device of claim 1, wherein the patient-ventilator asynchrony monitoring method further includes:
determining a proposed adjustment to the mechanical ventilation to reduce or eliminate the patient-ventilator asynchrony events detected by the applying of the patient-specific ML component;
wherein the display device is further configured to display the proposed adjustment.

6. The device of claim 1, further comprising:
a posture sensor configured to detect a posture of the associated patient as a function of time during the mechanical ventilation;
wherein the training further uses posture measurements received from the posture sensor to train the patient-specific ML component to detect patient-ventilator asynchrony events by analyzing ventilation waveform data and posture measurements.

7. The device of claim 1, wherein the ML component is further trained using imaging data of the associated patient.

8. The device of claim 7, further comprising:
an imaging device configured to acquire the imaging data.

9. The device of claim 1, wherein the indication of patient-ventilator asynchrony events comprises an asynchrony index; and
wherein the asynchrony index is displayed on the display device.

10. The device of claim 9, wherein the patient-ventilator asynchrony monitoring method further includes:
calculate the asynchrony index as a ratio of a number of asynchronous breaths of the patient in labelled waveforms over a predetermined time period and a total breath count of the patient over the predetermined time period.

11. The device of claim 1, wherein the patient-ventilator asynchrony monitoring method further includes:
determining one or more optimized settings of the mechanical ventilator based on the detected patient-ventilator asynchrony events; and
displaying the one or more optimized settings of the mechanical ventilator on the display device.

12. The device of claim 1, wherein the patient-ventilator asynchrony monitoring method further includes:
determining one or more optimized settings of the mechanical ventilator based on received imaging data; and
displaying the one or more optimized settings of the mechanical ventilator on the display device.

13. The device of claim 11, wherein the patient-ventilator asynchrony monitoring method further includes:
controlling the mechanical ventilator by adjusting settings of the mechanical ventilator with the determined one or more optimized settings.

14. The device of claim 13, wherein the patient-ventilator asynchrony monitoring method further includes:
tracking a progression of a patient-ventilator interaction; and
predicting a future patient-ventilator asynchrony event based on the tracking.

15. A mechanical ventilation method comprising:
receiving ventilation waveform data during mechanical ventilation of an associated patient;
detecting initial patient-ventilator asynchrony events during a training period of the mechanical ventilation by analysis of measurements of the associated patient acquired during the training period;
training a machine learning (ML) component to analyze ventilation waveform data to detect patient-ventilator asynchrony events using the ventilation waveform data received during the training period with labels indicating the initial patient-ventilator asynchrony events, the trained ML component forming a patient-specific ML component that is specific to the associated patient;
applying the patient-specific ML component to the ventilation waveform data received after the training period to detect patient-ventilator asynchrony events occurring after the training period; and
displaying an indication of patient-ventilator asynchrony events detected by the applying of the patient-specific ML component.
